(19) United States
(12) Patent Application Publication
(10) Pub. No.: US 2010/0130944 A1
(43) Pub. Date: May 27, 2010

(54) FLOW CONTROL DEVICES FOR OPHTHALMIC SURGERY

(76) Inventor: Douglas E. Music, Wildwood, MO (US)

Correspondence Address: Bausch & Lomb Incorporated, One Bausch & Lomb Place, Rochester, NY, 14604-2701, US

(21) Appl. No.: 12/275,898

(22) Filed: Nov. 21, 2008

(51) Int. Cl. A61M 39/00

(52) U.S. Cl. 604/246

(57) ABSTRACT

A flow control device for providing flow resistance to a surgical handpiece during ophthalmic surgery. The flow control device includes a chamber having two sides in parallel separated by a height, an inlet port extending tangentially from the chamber in parallel with the two sides for coupling to a surgical handpiece, and an outlet port extending from about the center of the chamber perpendicular to the two sides for coupling aspiration tubing.

### BACKGROUND

1. Field

The present disclosure directed to flow control devices for controlling fluid flow during ophthalmic surgeries.

2. Description of the Related Art

The statements in this section merely provide background information related to the present disclosure and may not constitute prior art.

In surgery, particularly eye surgery, surgery systems are commonly used to generate fluid flow to and from a surgical site. Irrigation provides fluid to the surgical site, while aspiration removes fluid and debris from the surgical site. In ophthalmic surgery, balancing the irrigation and aspiration is necessary for several reasons, including keeping the eye inflated and preventing collapse of the eye, which may cause serious damage.

During ophthalmic surgery, aspiration is used by a surgeon to evacuate debris from the surgical site. In phacoemulsification, for example, a surgical handpiece fitted with an ophthalmic needle is used to break apart a cataract and provide an aspiration path for evacuating cataract debris from the eye. The surgeon may employ aspiration to hold the cataract prior to applying ultrasonic energy to the cataract with the ophthalmic needle coupled to one end of the surgical handpiece. By holding the cataract, the ophthalmic needle may be partially or wholly occluded, often resulting in pressure building through a fluid path from the surgery site to the surgery system housing an aspiration pump and collection reservoir. Debris from the cataract may also partially or wholly occlude the ophthalmic needle. As pressure in the infusion fluid path increases during occlusion, fluid behind the occlusion is aspirated creating an ever increasing vacuum level. When the occlusion is removed, an inrush of fluid from the eye may cause an imbalance in irrigation/aspiration sufficient to cause damage to the eye. In order to prevent or at least minimize the chances of the damage resulting from occlusion, it is desirable to control fluid aspiration flow by increasing flow resistance to aspiration between the surgical handpiece and the surgery system. Increasing flow resistance also generally permits a higher level of vacuum to be maintained that, in turn, provides for a greater level of purchase or followability of the cataract to the needle, which is desirable to the surgeon.

### BRIEF DESCRIPTION OF THE DRAWINGS

The drawings described herein are for illustration purposes only and are not intended to limit the scope of the present disclosure in any way.

FIG. 1 is a perspective view of a flow control device according to the present disclosure;

FIG. 2 is an elevation view of the a flow control device of FIG. 1;

FIG. 3 is a cross-sectional view of the flow control device of FIG. 2 along line 3-3;

FIG. 4 is an elevation view of the a flow control device of FIG. 1 along line 4-4;

FIG. 5 is a perspective view of a surgical handpiece assembly including a flow control device according to the present disclosure; and

FIG. 6 is a cut-away perspective new of another flow control device according to the present disclosure.

### DETAILED DESCRIPTION OF THE PREFERRED EMBODIMENT

The following description is merely exemplary in nature and is not intended to limit the present disclosure, application, or uses.

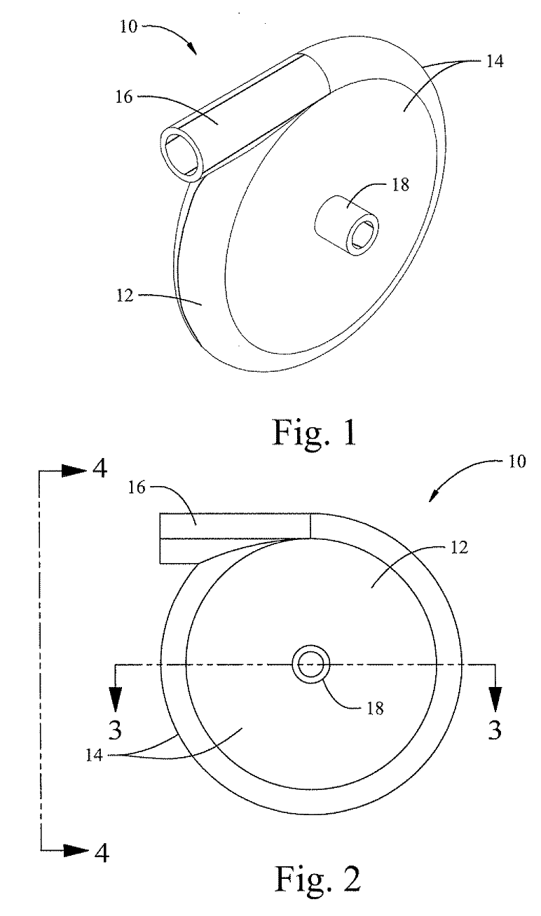

According to one embodiment of the present disclosure, a flow control device 10 is illustrated in FIG. 1. The flow control device 10 includes a chamber 12 having two sides 14 in parallel separated by a height, an inlet port 16 extending tangentially from the chamber in parallel with the two sides, and an outlet port 18 extending from the center of the chamber perpendicular to the two sides. In use, the inlet port 16 couples to a surgical handpiece, and the outlet port 18 couples aspiration tubing for transporting fluid between the surgical handpiece and a collection reservoir in an ophthalmic surgery system. The flow control device 10 helps maintain intraocular pressure in an eye by controlling aspiration fluid flow from the eye by increasing aspiration fluid flow resistance.

The inlet port 16 defines an opening, and the outlet port 18 defines an opening. The cross-section area of the opening of the outlet port 18 is preferably at least equal to the cross-sectional area of the opening of the inlet port 16. In this manner, any debris entering the flow control device 10 via the inlet port 16 is able to exit the flow control device 10 through the outlet port 18 such that a potential for clogging fluid flow through the flow control device 10 is eliminated. It should be appreciated that a cross-sectional area of an opening of an inlet port or an outlet port may also be selected to provide a particular flow rate through a flow control device.

FIG. 2 shows an elevation view of the flow control device 10, which defines a generally circular cross-section in parallel with sides 14. It should be appreciated that other cross-sectional shapes may be employed to affect flow resistance through flow control devices according to other embodiments of the present disclosure. For example, a chamber of a flow control device may define an ovular, a rectangular, or a triangular cross-section.

The flow control device 10 is formed from any medical grade material including metal or plastics. A different type of material may be included in a different flow control device for various reasons, such as cost, flexibility, manufacturability, suitability for use in medical procedures, etc.

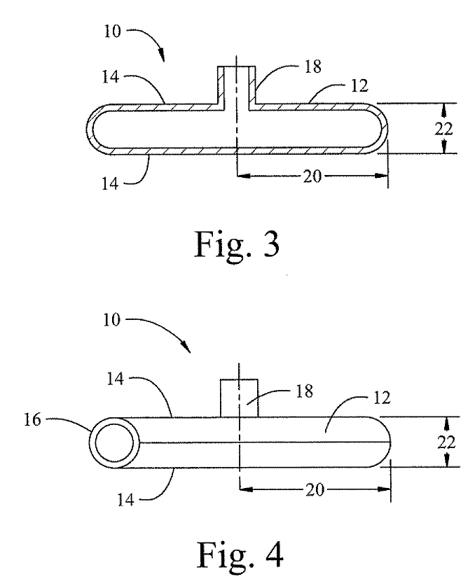

FIG. 3 shows a cross-section view of the flow control device 10 through line 3-3 in FIG. 2. As shown, the outlet port 18 extends from the center of the generally circular cross-section of the chamber 12. It should be appreciated that in other embodiments, an outlet port may offset from the center of the cross-section of the chamber. FIG. 3 further illustrates that the chamber 12 is hollow. By being hollow, flow resistance through the flow control device 10 is generally defined by the shape and dimensions of the chamber 12, as well as relative locations of the inlet port 16 and the outlet port 18. In other embodiments, it should be appreciated that a divider, a partition or another impingement to fluid flow may be included in a flow control device to increase or decrease flow resistance through the flow control device.

The generally circular cross-section includes a radius 20 denoted in FIG. 3. The radius 20 may be selected in combination with a height 22 of the chamber 12, referred to above. For example, a radius of a chamber having a generally circular cross-sectional may be at least two times a height of the chamber in at least one embodiment. It is desirable that the radius 20 be greater than the height 22 thereby providing a ratio of the radius to the height greater than 1. Generally, for a flow control device, according to the present disclosure, as the ratio increases, flow resistance between an inlet port and an outlet port also increases.

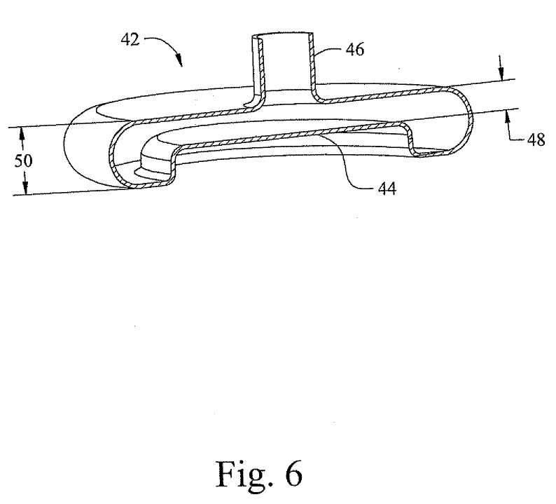

Flow resistance is generally dependent on the particular dimensions of the flow control device 10. Specifically, the ratio of the radius 20 to the height 22. For example, the radius 20 may be about 15 mm and the height about 1.5 mm. Therefore the ratio of the radius 20 to the height 22 is 10:1. The greater the ratio, the higher the flow resistance at any given flow rate. The higher the flow rate, the greater the resistance. With these two relationships, it is possible to select both absolute sizes and the ratio to achieve the desired resistance curve in relation to flow. It is believed that the physics creating resistance is primarily the conservation of angular momentum causing the flow to rotate ever faster as the fluid moves toward the center exit port. This spin produces shear forces between advacent fluid segment as well as flow resistance with the chamber walls. The inlet port and outlet port sizes are suitable to couple to known aspiration ports of the surgical handpieces. The height 22 can be considerably smaller than the inlet port if a clear path about the circumference of the device is at least the size of the inlet port. See discussion of FIG. 6 below. This allows particles to revolve in the fluid but not get lodged in the space created by the parallel walls separated by the device height 22. It should be appreciated that one or move of the dimensions may be increased or decreased for different flow resistances, other types of surgical handpieces and/or aspiration tubing, or other reasons related to performance, use or aesthetics.

FIG. 4 shows an elevation view of the flow control device 10 taken along line 4-4. As illustrated, the diameter of the inlet port 16 is substantially equal to the height 22 of the flow control device 10. In some embodiments, it is desirable that a height of a chamber be at least substantially equal to an aspiration port of a handpiece or aspiration tubing coupled to an inlet port of the flow control device. Accordingly, it should be understood that different size and shapes of inlet ports and chamber may be employed in a flow control device depending on a particular handpiece or aspiration tubing of an implementation.

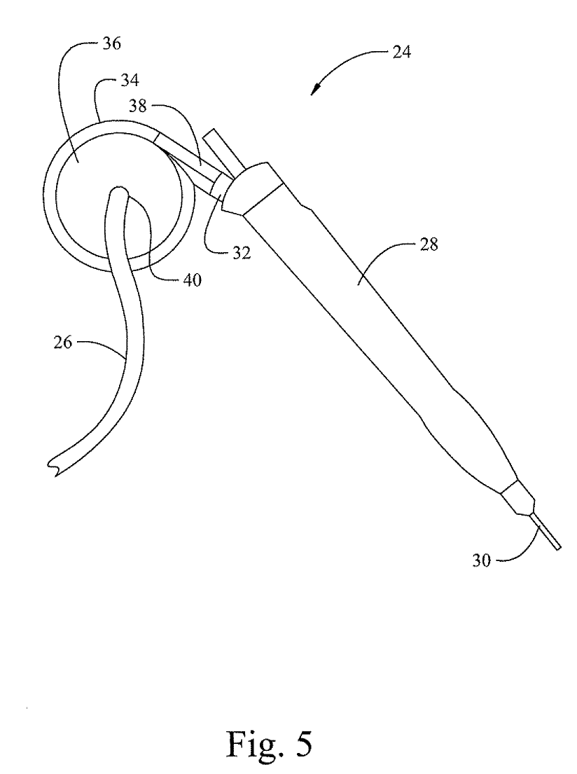

FIG. 5 shows a surgical handpiece assembly 24. The surgical handpiece assembly 24 includes aspiration tubing 26 for coupling to a surgery system (not shown) and a surgical handpiece 28 having an ophthalmic needle 30 and an aspiration port 32. The surgical handpiece 28, in combination with the aspiration tubing 26 and the surgery system (not shown), is configured to transport aspiration fluid exiting the aspiration port 32 and debris from a surgical site during a phacoemulsification surgery procedure.

The surgical handpiece assembly 24 also includes the flow control device 34. The flow control device 34 includes a chamber 36, an inlet port 38, and an outlet port 40. The outlet port 40 is connected to the aspiration tubing, and the inlet port 38 is coupled to the aspiration port 32 of the surgical handpiece 28. The flow control device 34 is preferably coupled between to the surgical handpiece 28 aspiration port 32 and aspiration tubing 26. In other embodiments, a flow control device may be coupled at a different point along a fluid path between a surgical handpiece and a surgery system. Accordingly, a flow control device may be connected directly to an aspiration port of a surgical handpiece or via a length of aspiration tubing.

The inlet port 38 includes an opening with a cross-sectional area, and the aspiration port 32 of the surgical handpiece 28 also includes an opening with a cross-sectional area. It is preferable for the cross-sectional area of the opening of the inlet port 38 to be greater than the cross-sectional area of the opening of the aspiration port 32, such that inlet port is configured to matingly connect to aspiration port 32. In this manner, any debris transported through the aspiration port 32 likely flows through the inlet port 38 without clogging and thereby interrupting fluid flow through the flow control device 32. The outlet port 40 also includes an opening with a cross-sectional area. Similarly, it is preferable for the cross-sectional area of the opening of the outlet port 40 be at least equal to the cross-sectional area of the opening of the inlet port 38.

FIG. 6 shows a cut-away perspective view of another flow control device 42. Device 42 functions and is constructed similarly to device 10 described above. The difference between device 10 and device 42 is central indented portion 44 opposite outlet 46. Central portion 44 in the chamber side opposite outlet port 46 and the chamber side with the outlet port 46 define a central height 48 that is much smaller than the height 50 separating the two chamber sides. Having central height 48 be much smaller than height 50, allows device 42 to achieve dimension ratios of radius to height (as discussed above) that would be difficult to achieve with device 10. This is because without the overall height 48, the radius of device 10 would be required to be so large as to be impractical to use in surgery. Placing portion 44 centrally relative to outlet 46 allows aspirated particles to flow about the periphery having height 50 without clogging the space defined by height 48 and without requiring a filter before fluid enters device 42.

Certain terminology is used herein for purposes of reference only, and thus is not intended to be limiting. For example, terms such as “upper,” “lower,” “above,” “below,” “top,” “upward,” and “downward” refer to directions in the drawings to which reference is made. Terms such as “front,” “back,” “rear,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import.

Although several aspects of the present disclosure have been described above with reference to aspiration during ophthalmic surgeries, it should be understood that various aspects of the present disclosure are not limited to aspiration during ophthalmic surgeries, and can be applied to a variety of other ophthalmic surgical procedures and methods.

By implementing any or all of the teachings described above, a number of benefits and advantages can be attained including improved reliability, reduced down time, elimination or reduction of redundant components or systems, avoiding unnecessary or premature replacement of components or systems, and a reduction in overall system and operating costs.

1. A flow control device for providing flow resistance to a surgical handpiece during ophthalmic surgery, the flow control device comprising:
a chamber having two sides in parallel separated by a height;
an inlet port extending tangentially from the chamber in parallel with the two sides for coupling to a surgical handpiece; and
an outlet port in one of the chamber's two sides and extending from about a center of the chamber perpendicular to the two sides for coupling to aspiration tubing.

2. The invention of claim 1, wherein the height is substantially equal to a diameter of the inlet port.

3. The invention of claim 1, wherein the chamber defines a generally circular cross-section in parallel with the two sides.

4. The invention of claim 3, wherein a radius of the generally circular cross-section is at least two times the height of the chamber.

5. The invention of claim 1, wherein a radius of the two sides is greater than the height such that a ratio of the radius to the height is greater than 1.

6. The invention of claim 1, wherein a central portion in the chamber side opposite the outlet port and the chamber side with the outlet port define a central height that is smaller than the height separating the two chamber sides.

7. A surgical hand piece assembly for ophthalmic surgery, the surgical handpiece assembly comprising:
a surgical handpiece having an aspiration port;
aspiration tubing for transporting aspiration fluid exiting the aspiration port; and
a flow control device coupled between the aspiration pod of the surgical handpiece and the aspiration tubing, the flow control device including a chamber having two sides in parallel separated by a height, a inlet pod extending tangentially from the chamber in parallel with the two sides, and an outlet port in one of the chamber's two sides and extending from about a center of the chamber perpendicular to the two sides.

8. The invention of claim 7. wherein the flow control chamber is positioned adjacent to the surgical handpiece.

9. The invention of claim 7, wherein the chamber defines a generally circular cross-section.

10. The invention of claim 9, wherein the generally circular cross-section includes a radius such that increasing a ratio of the radius to the height of the chamber increases flow resistance between the inlet port and the outlet port.

11. The invention of claim 8, wherein the height of the chamber is substantially equal to a diameter of the inlet port.

12. The invention of claim 10, wherein the ratio is greater than 1.

13. The invention of claim 7, wherein the inlet port is coupled to the aspiration pod of the surgical hand piece and the outlet port is connected to the aspiration tubing.

14. The invention of claim 13, wherein a cross-sectional area of an opening in the outlet port is at least equal to the cross-sectional area of the opening in the inlet port.

15. The invention of claim 7, wherein a central portion in the chamber side opposite the outlet port and the chamber side with the outlet port define a central height that is smaller than the height separating the two chamber sides.
